(19) United States
(12) Patent Application Publication
(10) Pub. No.: US 2022/0044697 A1
(43) Pub. Date: Feb. 10, 2022

(54) COMPUTERIZED SYSTEM AND METHOD FOR EVALUATING A PSYCHOLOGICAL STATE BASED ON VOICE ANALYSIS

(71) Applicant: CONNECTALK YEL LTD, Ramat Gen (IL)

(72) Inventors: Yehuda KAHANE, Herzlia (IL); Ernesto Sholomo KORENMAN, Raanana (IL); Liora WEINBACH, Herzliya-Pituach (IL)

(21) Appl. No.: 17/291,003

(22) PCT Filed: Nov. 9, 2019
(86) PCT No.: PCT/IL2019/051222

§ 371 Date: May 4, 2021

Related U.S. Application Data
(60) Provisional application No. 62/758,584, filed on Nov. 11, 2018.

(51) Int. Cl. G10L 25/63; G10L 25/78; G10L 21/0232

(52) U.S. Cl. CPC G10L 25/63; G10L 2025/783; G10L 21/0232; G10L 25/78

(57) ABSTRACT

A computerized method and system for analyzing and evaluating a psychological state of at least one user by operating a voice analysis system, the voice analysis system comprising a voice analyzer processor, comprising: tuning a user's voice by playing at least one beep sound followed by silence; receiving and recording at least one voice measurement from the user; utilizing the voice analyzer processor for automatically analyzing the voice measurements to evaluate psychological state of the user; wherein the voice analysis is based on harmonic analysis of the voice measurements; and providing the user, by visually indicating upon a display, with a feedback related to the user's psychological state.

### INTRODUCTION

Disclosed herein are a computerized system and method for evaluating a state of mind of a user based on objective parameters and optionally leading him though an emotional process in order to guide him to use a different, more rational, state of mind which consequently improves his ability to manage his emotions. More specifically, the present invention relates to a computerized system and method for evaluating a state of mind of a user utilizing a voice analysis.

### BACKGROUND

There are several communication mechanisms that are known in the art. A subject selects a specific communication mechanism in compliance with environmental conditions at a given situation. Thus, the role of speech is to provide feedback for two possible situations in the person-to-person relationship and person-environment relationship: (a) a conscientious, empathic, responsible, unifying, and conciliatory speech as a fruit of advanced communicative skills and echoing the correlation between a person and cohesive environmental signals. (b) mechanical, impulsive, artificial, offensive, and divisive speech echoing the lack of advanced and profitable communication, and a functional disconnection between a person and his proper hierarchical environment as sapiens.

Research shows that human reaction and/or verbal communication is typically controlled by the unconscious parts of the brain. These parts are known as the ancient parts of the brain and are driven by automatic survival instincts. All living species share that type of communication mechanism. Humans beings, as opposed to other living species, have the ability to use advanced verbal communication. The human speech mechanism is controlled either by the ancient parts of the brain, namely instinctive reactions, and by the frontal lobe of the brain that controls the reasonable and conscious communication.

The speech mechanism is managed by a natural neural switch that transfers control from the ancient parts of the brain to the advanced cognitive and conscious speech center and vice versa. The upper speech center distinguishes human beings from the animal world. The activation of the switch blocks the primitive and automatic speech and replaces it by an alternative humane, intelligent and moral conscious type of speech. This cognitive system manages any language and its recognition enables a person to be aware of her/his unconscious behavior.

A substantial part of human discourse is still controlled by the ancient parts. Switching control to the frontal lobe shifts the discourse to a co-operative level that avoids doing harm to others and at the same time brings into action strong self-healing powers.

To be able to communicate effectively and accurately, one must be aware of its own emotions and state of mind. If for example, a person is feeling stressed or unwell, he won't be able to shift the conversation to the co-operative level and therefore it would be better for him to defer having any important conversations. Reducing stress levels can help interact more positively and effectively with others. Being aware of one's state of mind and switching to intelligent rational type of speech is a technique that should be taught and spread around the world. Such technique will have impact over many fields such as psychology, sociology, political science, social work, marketing, management, decision making, education, medicine, analyzing art media, literature, movies, press coverage and news media, mass communication, social media, etc. When significant populations groups around the world exercise their communication skills, personal, public and multi-cultural discourse will be upgraded and will promote understanding and cooperation. This will have a profound positive impact on the welfare of the individual and of the whole human society. Spreading the technique to the entire world can result substantial economic gains (e.g., reduced defense spending and health budgets). This can easily and quickly be done worldwide.

Thus, there is a need to identify and analyze a human's state of mind and guide him through an emotional process to improve his communication skills. This is the basis for the emergence of a superior kind of communication abilities. A simple technique to quickly teach a user how to activate the switch has been developed and tested. The goal of this invention is to transform the unconscious into a collective consciousness frame and to instill a common, visual, casual universal language to deepen one's understanding of himself/herself and others. One prominent advantage is that the present invention designed to be accessible to all at all ages and realizing a social vision of equal opportunities.

The disclosed invention herein, refers specifically to the use of a computerized tool that applies a simple and user-friendly technique, utilizing a computerized voice analysis to improve for example, a human's communication skills, a human's ability to regain control over his intense emotions, etc. Moreover, the present invention uses an objective tool based on voice recordings evaluation to analyze the user's state of mind and further the improvement or the change of the user's state of mind, relying on an objective analysis, optionally in combination with the user's input.

These and other features and advantages of the present invention will be explained and will become apparent through the description of the invention and the accompanying drawings that follow.

### SUMMARY

The invention may be embodied as a computerized method for analyzing and evaluating a psychological state of at least one user by operating a voice analysis system, which comprises a voice analyzer processor, said method comprising several stages, comprising: tuning the user's voice by playing at least one beep sound, optionally adjusted a natural resonance frequency, followed by silence, receiving and recording at least one voice measurement at the length of at least 0.25 seconds, utilizing the voice analyzer processor for automatically analyzing the voice measurements to evaluate psychological state of the user; wherein said voice analysis is based on harmonic analysis of the user's voice measurements, and providing, by visually indicating upon a display, with a feedback related to the psychological state of the user.

Further, each voice measurement is recorded if the decibel level of the user's voice is at least 70 dB. Optionally the method, according to the first aspect of the invention, automatically detects the ambient background noise level and sets the threshold intensity of the voice measurement to be at least 0.5 dB above the ambient background noise level.

The evaluation of the psychological state of the user, provided herein is performed based on harmony analysis of the voice measurements, based on at least one natural resonance, such as the Schumann's Resonances, or any combination thereof. The method embodied herein, is operable offline or through a network comprised of the internet or a local area network, and the voice measurement is received from the user's electronic communication device, including a cellular phone, a personal computer, a tablet, and a personal digital assistant.

According to another aspect of the present invention, the method herein may evaluate the psychological state of a user with and without the effect of the presence of another person, therefore the method further comprises the following steps, this time under the effect of the other's person presence: tuning the user's voice by playing at least one beep sound followed by silence; receiving and recording at least one new voice measurement; utilizing the voice analyzer processor for automatically analyzing the new voice measurements to evaluate the psychological state of the user based on harmonic analysis; comparing the evaluations of the voice measurements recorded with and without the another person's presence, that were processed by the voice analyzer processor; evaluating the influence the other person has on the user; and providing with a feedback related to the results of that comparison.

According to another aspect of the present invention, the term ‘user’ refers to a plurality of users and therefore the method herein comprising receiving and recording at least one voice measurement that is composed from a plurality of sounds, that are simultaneously produced by a group of people, and thereby evaluating the psychological state of that group of people together.

According to another aspect of the present invention, the term ‘user’ refers to two users and therefore the method herein further comprising: tuning second user's voice by playing at least one beep sound followed by silence; receiving and recording at least one voice measurement from the second user; utilizing the voice analyzer processor for automatically analyzing the voice measurements of the second user to evaluate his psychological state, based on harmonic analysis of the voice measurements; comparing the evaluations of the two users; evaluating the degree of complementarity and harmony between them; and providing with a suitable feedback.

According to another aspect of the present invention, the method herein is providing a computerized emotional treatment for improving the psychological state of the user resulting in his ability to cope with his emotions, and in order to evaluate the psychological state enhancement in at least two different events and therefore the method herein further comprising: receiving the user's input indicating his pre-treatment psychological state, that is selected from a pre-defined list of optional psychological states; tuning the user's voice by playing at least one beep sound followed by silence; receiving and recording at least one voice measurement; utilizing said voice analyzer processor for automatically analyzing the voice measurements to evaluate pre-treatment psychological state of the user; automatically initiating the computerized emotional treatment, that is adjusted to the pre-treatment psychological state, by retrieving media files from a database of media files related to the computerized emotional treatment; optionally that treatment is provided in response to either the user's input, or to the evaluation of his pre-treatment psychological state; or in response to any combination thereof. The method according to hos aspect further comprises tuning the user's voice; receiving and recording at least one new voice measurement; further utilizing the voice analyzer processor for automatically analyzing the new voice measurements to evaluate post-treatment psychological state of the user; comparing the evaluations of the pre and post-treatment voice measurements; evaluating the psychological state enhancement based on that comparison; and providing the user with a feedback related his psychological state enhancement.

Optionally, the method further comprises receiving the user's input referring to the level of his feeling of discomfort, that is selected from a pre-defined list, before and after the computerized emotional treatment. The discomfort feeling is represented in numerical values from 1 to 10, that correlates with a percentage value of maximal protentional of human intelligence. The method further compares the before and after inputs and evaluates the psychological state enhancement based on said comparison.

Optionally, the comparison stage is performed multiple times, taking into account voice measurements that are received in different events including before, during or after the computerized emotional treatment stage, evaluating psychological state enhancement based on each comparison.

The invention may additionally be embodied as a voice analyzer processor for processing and implementing a computerized method for performing a harmony analysis of a voice measurement, that voice analyzer is configured to: receive and record at least one voice measurement from at least one user; define a sampling rate for each voice measurement; optionally the sampling rate is calculated based on a natural resonance; automatically calculate fast Fourier transform (FFT) spectrum values associated to each voice measurement, based on that sampling rate; perform plurality of corresponding calculations, thereby the voice analyzer is further configured to: automatically calculate an entropy value that is characterising said FFT spectrum values, based on probability analysis; construct harmonic frequency based on peak values of said FFT spectrum values; construct dis-harmonic frequency based on frequency peak values of said FFT spectrum values; and, automatically calculate variability of said harmonic or dis-harmonic frequency averaged values; or automatically calculate the ratio between the harmonic frequency averaged value and the dis-harmonic frequency values; identify correspondences between the frequency peak values of the FFT spectrum values or their average value, to the golden proportion, in a deviation of up to 20% in reference to the golden proportion; separately normalize the results of each corresponding calculations into one parameter ranging from 0 to 1, relating to each voice measurement; and unify the parameters, each refers to each voice measurement, into one final parameter; wherein the final parameter is designed to characterize a harmony degree in the user's voice measurements, thereby reflecting his psychological state.

Optionally, the FFT spectrum values are calculated based on a filtered voice measurement, namely omitting recognizable formants and elevated peaks of frequency in the voice measurement and replacing them by their mean value.

According to another aspect of the invention, the term ‘user’ refers to two users and therefore the voice analyzer processor is further configured to calculate two separate final parameters, one referring to one user and the other referring to a second user; and calculate the ratio between these two final parameters, wherein that ratio is designed to characterize the degree of complementarity and harmony between these two users.

According to another aspect of the invention, the voice analyzer processor is further configured to filter the FFT spectrum values by disregarding FFT spectrum values that exceed their mean value or by omitting all FFT spectrum values that exceed their mean value and replacing them by said mean value. In another embodiment, the voice analyzer processor is further configured to automatically calculate the logarithm values of each of the FFT spectrum values, and thereby the filtration of the FFT spectrum values is performed on their logarithmic values. According to another aspect of the invention, the voice analyzer processor is further configured to automatically calculate statistical parameters based on the logarithmic values of the FFT spectrum values, such as average, quartiles, standard deviations and the ratio values of any combination thereof.

According to another aspect of the invention, the voice analyzer processor is further configured to create a general database, storing the statistical parameters of variety of users, for further analytic and statistical purposes, and comparing the statistical parameters of the user with corresponding statistical parameters stored in that general database.

According to another aspect of the invention, the voice analyzer processor is designated to evaluate the user's psychological state enhancement in at least two different events, based on a harmony analysis of his voice measurement, and therefore is further configured to: separately normalize the results of each corresponding calculations into one pre-parameter ranging from 0 to 1, relating to each voice measurement recorded before an emotional treatment, as initiated by the computerized method; separately normalize the results of each said corresponding calculations into one post-parameter ranging from 0 to 1, relating to each voice measurement recorded after an emotional treatment, as initiated by said computerized method; unify the pre-parameters, each refers to each the voice measurement recorded before the emotional treatment, into one pre-final parameter; wherein the pre-final parameter is designed to characterize the harmony degree in the user's voice measurements recorded before the emotional treatment, thereby reflecting the user's psychological state before the emotional treatment; unify said the post-parameters, each refers to each voice measurement recorded after the emotional treatment, into one pre-final parameter; wherein the post-final parameter is designed to characterize the harmony degree in the user's voice measurements recorded after the emotional treatment, thereby reflecting the user's psychological state after the emotional treatment; and calculate the ratio between the pre-final parameter and post-final parameter, wherein said ratio is designed to evaluate the user's psychological state enhancement. Optionally, the voice analyzer processor is further configured to establish a significance degree of the psychological state enhancement based on probability analysis.

The invention may additionally be embodied as a system for processing, analyzing and evaluating a psychological state of at least one user, comprising: a computer software, interacting with associated peripherals; a communication device, selected from: a cellular phone, a personal computer, a tablet, and a personal digital assistant, hosting the computer software, the computer software is configured to receive information from the user; a database storing and analyzing the information; wherein the computer software is configured to utilize a voice analyzer processor, which is configured to process and analyze at least one voice measurement that was received and stored in the database; to evaluate a psychological state of said user, based on harmonic analysis of his voice measurements; and to provide the user with a feedback related to the evaluation of psychological state, displayable upon a user display.

Optionally, the system embodied herein is operable offline or through a network comprised of the internet or a local area network, wherein the system utilizes a server in communication with the user's communication device, that may be utilized to communicate with the server; the database is configured to store and analyze the information received from the server; and the server is configured to provide the feedback to the user's communication device for display upon a user display associated therewith.

Embodiments of the present invention are described in detail below with reference to the accompanying drawings, which are briefly described as follows:

### BRIEF DESCRIPTION OF THE DRAWINGS

The invention is described below in the appended claims, which are read in view of the accompanying description including the following drawings, wherein:

FIG. 1 shows a table of major features of consciousness levels

### DESCRIPTION

The invention summarized above and defined by the claims below will be better understood by referring to the present detailed description of embodiments of the invention. This description is not intended to limit the scope of claims but instead to provide examples of the invention.

In recent years science gained better understanding of human speech and discovered the natural neural switch that controls the transfer from automatic to conscious speech. The invention disclosed herein refers to a computerized system and method for identifying a state of mind of a user based on voice analysis, operated by a voice analyzer, which is an objective and unbiased tool, in order to lead the user through an emotional process in order to guide him to use a different, more rational, state of mind. As a result, a mood enhancement effect is achieved, namely the user is taught to improve his state of mind which consequently improves for instance, his verbal communication skills.

Generally, the system and method of the disclosed invention are aimed to activate the switch between two types of speech communication. It teaches a user how to wake up a dormant and creates the ability to block primitive and automatic speech and replace it by an alternative humane, intelligent and moral conscious type of speech. These system and method are convenient for all ages, from early childhood and it does not require any prior knowledge. Study of the imaging of the brain confirms the existence of a brain structure and its five developmental stages. Methods of imaging that are based on the function of the brain cells sharpen the existence of the brain centers and the roles they play in human behavior. Observation of evolution by means of the science of brain isotope imaging leads to new understandings of inner and hidden processes and their external manifestations in the fields of biology, chemistry and brain.

The technology disclosed here can be applied in practical teaching, in various languages and with a variety of educational levels and all age groups, such as in undergraduate and graduate levels in universities, schools, elementary and secondary schools, kindergartens, special education as well as in special groups like parents, medical teams, communication courses (including but not limited to analysis of mass information, press coverage, news media, social media, art media, literature, movies, etc.), human resources and organizational consultants, management and leadership programs, courses in the community, violent people, prisoners, family treatments, judges and mediators, etc. People at all ages above four years old can easily and quickly learn to gain control on the switch using this invention.

Further this system and method of the disclosed invention are also may be used to allow a user to cope with his eating disorder, by leading the user through an emotional process in order to guide him to use more rational state of mind, thereby allowing him to regain control over his intense emotions that are the cause of that eating disorder.

As used herein the description the term ‘user’ is intended to refer to a single user or to a group of users, as may be applicable, unless explicitly stated otherwise.

Various embodiments of the invention generally seek to provide mood enhancement by use of a computerized system and method for further guiding a user through several emotional steps in order to improve his state of mind resulting for example, in enhancement of the user's verbal communication skills, in his ability to manage variety of emotional disorders, in his ability to regain control over his intense emotions, etc. Thus, the user of the herein disclosed invention learns how to activate his rational speech mechanism, rather than his instinctive one. The use of a computerized tool enables a daily use in learning the evaluation, feedback and assessment of self-improvement.

According to other embodiments of the invention, other implementations are also optional, as specified above. The common purpose of all these implementations is to improve the user's state of mind, thereby the user can, for instance improve his communications skills, manage his eating disorders, manage variety of emotional disorders, regain control over his intense emotions, etc.

According to some embodiments of the invention, the stage of the emotional treatment is optional. In these embodiments, the purpose of the disclosed system and method is to evaluate the psychological state of a user, without leading him through an emotional treatment.

Said method is performed by integration of three components of the system (1) an application residing on a mobile communication device. Not limiting examples of such device are iPad, laptops, mobile computers, desk computers with keyboard, desk computers with touch screens, a cellular phone (smartphone, such as iPhone®, smartphone using the Android® operating system); and/or (2) a website hosted by a server, coupled to a network; (Components (1) and/or (2) are referred herein as “the software”); and (3) a database that stores and analyzes data associated with the user, from either the above two components. In some embodiments of the invention, the software is operable offline or is operable to use a network connection to receive data and to perform various of functions on said data.

The invention may be embodied in multiple ways. Non-limiting example embodiments are discussed as follows:

In some embodiments of the invention, a user is guided to go through an emotional process, including several stages. The software first displays to the user an opening screen in which the goal of the software is introduced to the user. As above described, according to some embodiments of the invention, the goal of the software is to guide the user to reach an improved state of mind and activate his rational speech mechanism. The software presents to the user a start screen, acknowledging the user has an actual problem that he would like to solve with the help of the software. The software displays to the user several options to describe his current state of mind. Optional wordings are for example “I FEEL RESTLESS, I AM STRESSED, FRIGHTENED, HURT, UPTIGHT, CONFUSED, DESPERATE, HOPLESS”. The software then teaches the user that in order to obtain a solution to improve his current state of mind, the user should seek for the reasons to his negative emotions. In view of these reasons, the software operates an adjusted course of treatment in order to treat the stimuli factors that caused this situation from the first place. The emotional treatment implemented by the software is therefore in various embodiments designed to enhance the mood, state of mind, physical well-being, psychological state, or other such mood-related state of the user.

Lastly, the software is configured to urge the user to explore whether his emotions have changed in a positive way and whether his feelings were improved. The software, in addition to its goal to make a change in a user's state of mind, is configured to be very informative and guide the user through explanations of the emotional process step by step. Understating the psychological background is a substantial part of the emotional treatment and helps the user to succeed. Therefore, in some embodiments of the invention, the software includes informative materials along the process itself. In other embodiments of the invention, the software further encourages the user to persist the process and shows him linguistic mantras, for example: “I DO NOT ATTACK”, “I DO NOT BLAME”, etc. and various motivations that he could easily relate to in order to empower him as well as to strengthen him to go through the entire process with success. The information presented to the user, such as animations, video files, audio files, visual information, presentations, questions, guiding materials and all other elements or information that may be embodied within the software (also referred here as media files) defined the course of the emotional treatment. The course of the emotional treatment is typically under the control of the software, and can be varied according to the software's instruction, such as displaying media files that are adjusted to the user's current state of mind according to his own perception, as reflected by the user's elections at the beginning of the software's session. In some embodiments of the invention, any of these media files can be varied in pace, and the sounds accompanying them can be changed in pitch, volume, or in any other characteristics to make the presentation more relaxing.

The invention disclosed herein utilizes a voice analysis operable by a voice analyzer, in order to identify and to evaluate the state of mind of the user, and therefore it shows in an unbiased way whether the user's mood was actually altered or improved by the end of the session of the software. The voice analysis is conducted based on natural frequencies, identifying the harmonic level characterizing the user's voice.

It is studied that natural frequencies could affect humans in a variety of ways. Natural frequencies are frequencies that occur in nature, such as various periods or resonances calculated from planetary orbits. It is further studied that natural frequencies have significant emotional as well as psychological effects when presented to a person. Some frequencies have healing effects and they are used to address variety of illnesses. Other frequencies have emotional effects and they may be used to address mood enhancement. As said, the disclosed invention comprises a voice analyzer that is operable to identify the user's state of mind, in reference to natural frequencies. Natural frequencies include for example, brainwave frequencies that are associated with various emotional states. By coaxing a person's brainwaves to a certain frequency, an emotional state associated with that frequency, is achieved. Another example, is the Schumann's Resonances (ShR) that are a set of spectrum peaks in the extremely low frequency portion of the Earth's electromagnetic field spectrum. Schumann resonances are global electromagnetic resonances, excited by lightning discharges in the cavity formed by the Earth's surface and the ionosphere. The Schumann's Resonances may affect the frequency spectrum of human brainwaves rhythms and therefore may influence on a person's state of mind. The Schumann's Resonances frequencies start at 7.8 Hz and progress by approximately 5.9 Hz. (7.8, 13.7, 19.6, 25.5, 31.4, 37.3, and 43.2 Hz.) (also referred as the Schumann's Resonance elements). These frequency peaks appear to relate to known wave frequency elements in the electroencephalography (EEG), typically referred to as Alpha-Theta, SMR, Low Beta, Beta, High Beta and Gamma.

According to some embodiments of the invention, the software of the invention is configured to initiate an emotional treatment session wherein the software displays to the user at least four types of states of mind for his election. The user is requested to choose the most appropriate description to his current state of mind Not limiting examples are: “I MAKE UP MY MIND SOLELY, WITHOUT THE HELP OF ANYONE ELSE, I ACCUSE MYSELF, I AM ANGRY AND BLAME ANYONE ELSE FOR IT, I AM AFRAID AND AGGRESSIVE”. The user is then requested to proceed the process according to the elected emotion, he related to the most.

The software offers the user several paths (course of an emotional treatment), each complies with a specific state of mind. All processes aim to bring the user to another state of mind that would eventually activate his intelligent, rational speech mechanism.

The software then provides the user with explanations for his current mood and the harmful outcome it might lead to. The software teaches and trains the principles of the emotional treatment and helps to implement them to resolve domestic situations of unrest, stress, mood-swings, depression, anxiety, conflict resolution, decision making, etc. The software displays to the user information of his current mood, such as it is unhealthy and may cause the ancient speech mechanism to take control which might eventually harm relations, lead to loneliness, grief, heart-ache, alienation, etc. In some embodiments of the invention, the software further displays guidance and instructions in order to overcome this mood and switch to a conscious mode of the brain. For example, the software guides the user to follow a few simple steps, such as: (1) to couple the tongue to the upper palate to block any unconscious, impulsive speech mechanism, (2) defer any communication with others and avoid from verbally attacking anyone, (3) take deep breaths, (4) moving the eyes laterally to achieve desensitization, (5) focus on positive thoughts, and repeat an empowering mantra to help the user to connect to a positive energy. Other energetic-psychological techniques for inducing self-consciousness may be applied at this stage. It is explained to the user that performing said simple steps would help him to calm himself and switch his state of mind into more relaxed and rational one.

The method implemented by the disclosed invention helps a user to deal with complicated emotional situations. Further, said method offers a consistent and long-term avoidance from the impulsive and mechanical speech patterns, (mediated by archaic brain structures) and instead switch into a (frontal lobe mediated) speech that is in harmony with the environment. This process leads to the realization and expression of humane intelligence through conscious, unifying, and conciliatory speech.

Throughout the emotional treatment, the software displays to the user information of the emotional treatment, as may be embodied within the software's instruction. As above said, the software evaluates whether there is any improvement of the state of mind of the user, by analyzing the user's voice at least two times during the emotional treatment, which are before and after the treatment. At these times, the software disclosed herein further initiates a voice analyzer, thereby the software provides the user with individual quantitative feedback (also defined in the art as biofeedback) on his progress following the emotional treatment. The voice analyzer utilizes in various embodiments various devices, examples of such including a microphone, an antenna, a speaker, a screen that serves as media displayer and as an input receiver, keyboard. The term ‘media’ or ‘media files’ refers in this context to audio and/or video files and/or visual information and presentations that are under the control of the software, and are displayed or not-displayed according to the software's instruction.

Generally, according to some embodiments of the invention, the voice analyzer performs two steps of analysis: the first is performed before the emotional treatment and the second, after the emotional treatment. Through each stage, at least one voice measurement is recorded and analyzed according to a voice analysis method as will be disclosed hereinafter. Then, the voice analyzer compares the two results from pre and post treatment and evaluate the change in the user's mood before and after the emotional treatment session. Finally, the software presents a feedback to the user, based primary on the voice analysis or according to some embodiments based on both the user's elections through the emotional treatment and the voice analysis.

In other optional embodiments, the voice analyzer performs voice analysis without leading the user though an emotional treatment. In these examples, at least one voice measurement is recorded, the voice analyzer analyses and evaluates the user's mood, and the results are presented to the user.

### Recording a Voice Measurement

The software according to this invention, is configured to record the user's voice measurement at least two times during the emotional treatment: the first time, before the users starts the emotional treatment and the second time at the end of the emotional treatment. However, in other embodiments of the invention, additional voice measurements of the user can be recorded and analyzed along different stages of the emotional treatment. Optionally, in other embodiments of the invention, the software is configured to record the user's voice measurement, which is then analyzed and the evaluated state of mind of the user is evaluated and presented to the user, without leading him through an emotional treatment. According to another optional embodiment, the software is configured to further record a voice measurement of another user, and the voice analyzer compares the results of the two users, and present feedback as to degree of complementarity and harmony between them, based on the harmonic analysis of their voices. These kinds of modifications will readily occur to those skilled in the art and therefore are included in the scope of this invention.

The voice measurement is taken, according to the following stages:

### (1) Voice Tuning

First, the software presents the user with instructions before taking the user's voice measurements through a microphone. In other embodiments of the invention, the voice measurement is received through an antenna that is designed to receive radio waves. The user is requested to find a quiet place, in order to minimize background noise; to place the microphone at a distance of about 10 cm from his mouth; and press “START” when ready to start.

Next, the software of the invention, tunes the voice of the user by producing a several beep sounds are played through a speaker. According to some embodiments of the invention, at least two constant beep sounds are produced, wherein each beep lasts for up to half second and followed by up to half second of silence. Playing plurality of beep sounds heighten awareness, promote a feeling of relaxation, and cause other feelings of well-being. In other embodiments of the invention, the beep sounds are at least one note and preferably three notes, that are played in an adjusted frequency associated with a natural resonance, such as the Schumann's Resonances.

As the Schumann Resonances drive the harmonic pulse of life in general, then by exposing the user to said beep sounds, the user is psychologically harmonized to the Schumann's Resonances cycle. It is believed that this has a relaxing effect of the user's body and mind and the user's voice is tuned to its most harmonic expression.

According to one embodiment of the invention, the following sequence of several beep sounds are produced, optionally three, each for half a second, followed by up to half a second of silence, all based on the second element of the Schumann's Resonances (13.7 Hz): 219.20 Hz, 276.17 Hz and 328.43 Hz. In western music, the most common tuning system has been a twelve-tone equal temperament. The full musical octave is divided into 12 parts, all of which are equal on a logarithmic scale, with a ratio equal to the 12^{th} root of 2 (12√2≈1.05946). This resulting in smallest interval, 1/12 the width of an octave, which is called a semitone. That said, in order to calculate the frequencies of the beep sounds a musical scale (which is also referred herein as Equaled Tempered Scale) is built with the note A-1 value at a frequency of exactly 13.7 Hz. Higher notes in the scale are calculated by multiplying this value by the 12th root of 2 (12√2≈1.05946). For instance, following note: A#-1=13.7*1.05946=14.5146 Hz. Then, B-1=14.5146*1.05946=15.3777; C-1=16.2921 Hz; and so on. This calculation allows to find all higher notes in the musical scale, for instance A3=219.20 Hz; C#3=276.17 Hz; E3=328.43 Hz.

Typically, musical instruments are tuned to a standard pitch of 440 Hz, called A440, meaning one note, A, is tuned to 440 Hz and all other notes are defined as some multiple. The musical scale based on 13.7 Hz used in this disclosure is an alternative musical scale with A4=438.40 Hz. Similarly, other musical scales can be built in reference to other elements of the Schumann Resonance. For instance, a musical scale based on 7.83 Hz, which is the first element of Schumann Resonance, is achieved with A4=446.45 Hz.

### (2) Recording

In the next stage, the user is requested to produce a sound though the microphone. In some embodiments of the invention, the user is requested to produce a sound of a vowel. A vowel pronunciation enables not only to define an accurate personal voice signature of the user, but also to characterize minor changes in the voice measurement apart from the particular voice signature. When these changes are properly defined, parametrized and processed, the software according to the invention enables to qualify and quantify parameters such as harmony, entropy, musicality, natural order, environmental synchronization patterns and others in the user's voice, namely qualify and quantify consciousness and/or un-consciousness aspects of the user's mind in a high degree of accuracy.

The software records and stores a voice measurement when the decibel level of the user's sound has a pre-determined threshold intensity. The value of the threshold intensity is a variable that may be changed depending on the level of the ambient background noise in a particular environment. Ambient background noise in metropolitan, urbanized areas typically varies from 60 to 70 dB and can be as high as 80 dB or greater; In this embodiment of the invention the value of the threshold intensity may be about 75 dB. Quiet suburban neighborhoods experience ambient noise levels of approximately 45-50 dB, therefore in this embodiment of the invention the value of the threshold intensity may be about 60 dB. In other examples of the invention the value of the threshold intensity is about 70 dB. In another embodiment of the invention, the threshold intensity is automatically determined by the software, which detects the ambient background noise level and then set the threshold of the recording to ensure a good quality of the voice measurement, preferably at least 0.5 dB above the ambient background noise level.

According to the invention, the duration of a recording of the voice measurement required in order to provide assessment and feedback to the user is very short. In some embodiments of the invention, it is about 0.25 seconds. Nowadays, in order to perform a voice analysis, current known solutions require voice recording which are relatively long, (several minutes). One of the advantages of the disclosed invention, is that a comprehensive analysis of the voice can be achieved based on a very short voice recording, such as 0.25 seconds.

When the voice measurement is classified as natural, clear, effective and has a steady amplitude the software acknowledges the recording and store the voice measurement. If the recording is not successful, the user is asked to repeat the recording. The user is requested to repeat this stage for several times, such as three times in some embodiments of the invention. The voice measurements are stored and indexed as pre-treatment voice measurements (1, 2, 3, . . . ), when recorded before the emotional treatment and accordingly as post-treatment voice measurements (1, 2, 3, . . . ), when recorded after the emotional treatment. The results of the voice analysis are also stored.

In some embodiments, the user's voice measurements, and its voice analysis results are stored, such as in a user profile or in a server such that they can be referenced based on identification of the user. This enables to re-use this information and establish a personal database over time per user. In addition, it may also enable data analysis of a single user or a plurality of users. The software in this embodiment therefore tracks changes in the voice analysis results over time. Changes in voice analysis results are therefore not only observable as changes from a previous software's session, but are comparable against a time-weighted average of voice analysis results of the same user or against voice analysis results of another user or plurality of users. This data analysis is then may be used to improve the emotional treatment and change its content accordingly.

In other embodiments of the invention, the voice analysis is performed using single voice measurement of a plurality of users, producing sound together as a group. The software, in this example, is configured to analyze the degree of harmony in the voices of a group of people and even of whole populations. This application of the software may be applied in order to analyze harmonics in schools, sports stadiums and the like. Moreover, it allows a deeper understanding of the inter-relationship between humans. In further embodiments of the invention, the software tracks changes in the voice analysis results between two users that are interesting to explore the degree of complementarity and harmony between them, based on the harmonic analysis of their voices. Comparing voice measurements of more than two users is also included within the scope of this invention. The invention may also be applied to various purposes or embodiments, which are also within the scope of the present invention, and logical and other changes may be made without departing from said scope.

Finally, the software is configured to converts the voice measurements into audio files, such as .wav, .mp3 and the like.

### Voice Analysis

As said, the software is configured to analyze said voice measurements to identify the state of mind of the user, before and after an emotional treatment, based on several parameters. A detailed description of the voice measurement analysis is described hereinafter.

(1) Preparation of the Voice Measurement for FFT (Fast Fourier Transformation):

Mechanically, voice production involves the lungs, the vocal cords within the voice box, and the articulators (tongue, palate, lips, jaw etc.). The lungs produce adequate airflow and air pressure that provoke the oscillation of the vocal cords. The vocal cords then vibrate to use airflow from the lungs to create audible pulses that form laryngeal sound source. The muscles of the larynx adjust the length and tension of the vocal cords to ‘fine-tune’ pitch and tone. The articulators articulate and filter the sound emanating from the larynx (vocal cavity) and to some degree can interact with the laryngeal airflow to strengthen it or weaken it as a sound source.

That sound is transformed by the resonances of the pharyngeal, oral and nasal cavities. The position of the larynx, tongue, lips and jaw, as well as the tension of muscles around the vocal cavity modify the structure of the vocal “instrument” thus creating different resonance characteristics. For effective communication of meaning, the voice source, as a carrier for the selective spectral modification by the vocal cavity, contains harmonic energy across a large range of frequencies that spans at least the first few acoustic resonances of the vocal cavity.

When the vocal cavity remains open, a vowel sound is perceived. Each vowel is related to a particular positioning of the vocal structure and exhibit a particular spectral energy maximum in certain frequency ranges that correspond with the resonances of the vocal cavity present during their production. These spectral energy maxima are known as ‘formants’, that together with the fundamental frequency and its harmonics compose a full vocal spectrogram. In conversation, there are transitions from one sound to another with the vocal structure open, semi-closed or totally closed (for consonants and silences). The formant frequencies help the ear to identify the vowels and consonants thus discern the content of the conversation.

There is known scientific literature that studies the spectral peak of the formants, (the “Linear predictive coding” or “LPC”) according to the expected frequency formant for that vowel disregard of the fundamental frequency, its harmonics, extra unspecific formants between the expected ones, or other “spurious” extra formant specific for a particular speaker. The LPC is a method for signal source modelling in speech signal processing used for laboratory phonology, sociolinguistics and other linguists' interests. Unlike the LPC approach, in the disclosed invention, all specific or unspecific formants are taken into consideration, including all the frequencies and all the harmonics present. That way the full vocal spectrogram becomes a rich basis to quantify significant statistical features of the voice. The richer the voice in spectral peaks, the more vital the quality of the voice.

In view of the above, the voice analyzer employed in the disclosed invention, defines a particular sampling rate (SR) for each voice measurement. According to some embodiments of the invention, the sampling rate is calculated according to a given natural resonance value, such as the Schumann's Resonances. Calculating the sampling rate of a voice measurement, enables to calculate a Fast Fourier Transform (FFT) Spectrum. The term ‘FFT spectrum’ or ‘FFT’ in this context refers a computerized algorithm for converting a sequence of values into components of different frequencies. The value of the SR is calculated using the following equation 1: SR=Fq*N/4, wherein SR is sampling rate in Hz; Fq is the frequency of a natural resonance; N is the number of data values to be used to calculate the FFT spectrum.

For example, the voice analyzer is defined to set the value of Fq to be equal to 13.7 Hz, which is the value of the second element of the Schumann's Resonances and the value of N to be equal to 2048, then SR equals to 7014.4 Hz. The voice measurement is sampled at 7014.4 Hz. The sampling length of each period is 1/7014.4 sec=0.0001425638 sec. The voice analyzer calculates the FFT spectrum over 2048 data values (samples), to eventually receive a full FFT spectrum amplitude values. The audio files contain values of amplitude of sound intensity and the FFT spectrum values contain values of amplitude of frequency power in the spectral frequencies. As a result of this stage of the voice analyzing process, the amplitude values of the voice measurement as contained in the audio files, enable the calculation of the FFT spectrum values in precise frequencies of harmonics of the Schumann's Resonances element. Next, the frequential content of the voice measurement is compared to the natural resonance, as defined in the voice analyzer to be used in the above equation 1. Thus, identifying and quantifying distinctive peaks of formant frequencies of the voice measurement are based on above calculations and comparison. Said formant frequencies are derived not only from the architecture of the user's voice but also on psycho-physiological factors related to subconscious movements of the articulators (especially the subtle positions and tension of the tongue, the lips and other muscles) during voice production.

### Filtration and Noise Reduction

In some embodiments of the invention, the above FFT spectrum values are calculated based on a filtered voice measurement. An optional filtration may be used is omitting all recognizable formants and elevated peaks of frequency in the voice measurement and replacing them by their mean value.

Another example of filtration is noise reduction, in which the voice analyzer is configured to remove FFT spectrum values that considered to be ‘noise’. ‘Noise values’ are FFT spectrum values that exceed the average value of the FFT spectrum values. It is implied that formants of the FFT spectrum values are an example of noise values. In some embodiments of the invention, the noise values are disregarded and the result is filtered FFT spectrum values. In other embodiments of the invention the noise values are omitted and replaced by the average value of the filtered FFT spectrum values. The remaining values indicate the level of the vocal harmony evaluated in the voice measurement and thereby the voice analyzer enables to establish the degree of adaptation of the user's voice to the environmental. In some embodiments of the invention, the above stage of noise reduction of the voice analysis is performed on logarithmic values of the FFT spectrum values, as will be discussed in the following paragraph ‘spectral analysis’.

### Spectral Analysis:

In some embodiment of the invention, the voice analyzer employed in the disclosed invention, is configured to calculate statistical parameters of the FFT spectrum values.

First the voice analyzer calculates a logarithm of all and each of the FFT spectrum values. Thereafter the voice analyzer calculates statistical parameters of the logarithmic FFT spectrum values, such as: average (Avg), quartiles (Q), standard deviations (StDev) etc. According to some embodiments of this invention, it was found that these statistical parameters are good indicators of voice harmonies. For example, the outcome of the ratios: Q3/Q2, StDev/Q2 and StDev/Avg provide reliable quantitative markers (factors) to define voice harmonics and they are used in later calculations, as will be described thereafter. As mentioned, these parameters represent quantitative factors that are calculated by averaging of all other quantitative factors that reflect harmony.

The voice analyzer is configured to identify and quantify the influence of the natural resonance on the user's voice, as being reflected in the voice measurement. Further, the above equation 1 allows to evaluate the harmonics in the voice measurement and thereby to establish the degree of adaptation of the user's voice to the environmental resonances.

Following are carried out several parallel analyzing processes, all based on the FFT spectrum values received in this stage, relating to each voice measurement. The parallel stages are: (a) entropy analysis; (b) musical analysis; and (c) golden proportion analysis.

### (2A) Entropy Analysis:

In the next stage, the voice analyzer employed in the disclosed invention, is configured to calculate the value of entropy that characterised the FFT spectrum, which is a series of values (also referred here as time series).

Intuitively, there is some connection between uncertainty and probability, thus, the less probable an outcome is, the more uncertainty it has. Generally, entropy refers to disorder or uncertainty, thus entropy, as the level of disorder in a system is maximal in a series of random variables. In some embodiments of the invention, the voice analyzer calculates the Shannon Probability (ShP), according to which an entropy is the average rate at which information is conveyed by a stochastic source of data. The probability is therefore calculated based on the amount of information in a given time series.

In some embodiments of the invention, the Shannon Probability is calculated referring to a previous group of values of the FFT spectrum (for instance the last previous six (6) values). The more a time series is predictable based on its last previous group of values, (i.e. Probability (P) is large), its entropy is accordingly lower and its harmony is higher. The Shannon Probability is calculated for each value of the time series according to the following equation 2: ShP=((Avg/RMS)+1)/2 wherein Avg is the average of the previous six (6) values in a given time series; RMS is root mean square over the previous six (6) values.

In other embodiments of the invention, the voice analyzer may implement other statistics tools in order to present the entropy of a given time series, such as the approximate entropy (ApEn), which is used to quantify the amount of regularity and predictability of fluctuations over time series.

In some embodiments of the invention, the above analyzing process is implemented on FFT spectrum values which were calculated based on a filtered voice measurement or, in other examples the entropy analysis is implemented on filtered FFT spectrum values in which all the peaks of the FFT spectrum values are omitted, that optionally may replaced by the average value of the filtered FFT spectrum values, in order to perform the entropy analysis on the remaining low power values of the FFT spectrum values. These remaining values reflect how ‘predictable’ is the FFT. Thus, the value of entropy level is free from all formants of the voice measurement and its harmonies or disharmonies.

The voice analyzer at this stage is configured to identify and quantify the level of the vocal harmony in the voice measurement and thereby to establish the degree of adaptation of the user's voice to the environment.

### (2B) Musical Analysis:

Parallelly, the voice analyzer employed in the disclosed invention, is further configured to identify the level of the musical harmony in the voice measurement, based on the musical analysis of the FFT spectrum values.

The term ‘musical vocal harmony’ refers to a pleasing combination of different notes in a whole. Generally, harmonies are controlled by mathematical proportions covered in musical theory conventional principles. In the disclosed invention, a ‘musical vocal harmony’ is detected and analyzed by identifying a particular mathematic relationship between the frequencies of the different tones of the voice. Thus, the term ‘musical vocal harmony’ in this disclosure refers to a pattern of frequencies of the tones in the voice measurement that enable to teach of a rational state of mind the user is in.

Known in the art are musical therapeutic methods based on voice profiling which is implemented in a computerized frequency analysis using voice recordings for up to several minutes. That frequencies analysis (based on regular tuning musical scales) enables to quantify the emotional, biochemical and structural status of a patient. These methods treat the voice as an “holographic representation of the body” to assist in the management of health and wellness.

All parts of the body from the cellular level up to the very complex structures involved in emotional and mental processing functions at different frequencies. They are meant to work independently, yet as a whole they function in complete harmony. A person's voice represents all of the frequencies in the body; therefore, the voice is an ideal candidate for performing harmony analysis of a person. Generally, a healthy, emotionally balanced and mentally well-adjusted person hits all the notes of the musical scale when he speaks. Whereas the voice of an ill person or in terminal condition, is very restricted may consist of just a few notes.

According to the present methods of harmony analysis of a person's voice, long voice recordings are required, during which the effects of natural mechanisms of articulation, occlusion and breathing modification are reflected as the person pronounces full words, transitions and pauses.

As opposed to that, in the disclosed invention a short recording (about 0.25 seconds) of the user's voice is sufficient in order to achieve a complete harmony analysis of the user's voice. Further, the vocal harmony analysis is performed based on a natural resonance, rather than a regular musical scale.

According to some embodiments of the invention, at this stage of the voice analysis carried out by the voice analyzer, the vocal frequencies are being analyzed using a musical scale which is attuned to the environmental 13.7 Hz Schumann Resonance, using a sampling rate of 7014.4 samples/sec accordingly.

The voice analyzer is configured to construct musical harmonic frequency analysis based on all the formants of the FFT spectrum values rather than in a sequence of musical notes based on changes of the dominant frequency formant of the voice over longer periods as in singing a melody. Similarly, the voice analyzer is configured to construct musical dis-harmonic frequency based on all the formants of the FFT spectrum values.

There are two examples of musical harmony analysis implemented by the invention disclosed herein: (a) Musical Note variability analysis; and (b) Relative Power analysis.

### Musical Note Variability Analysis:

In this stage of analysis, the power of the various musical notes in all hearing octaves are integrated to show a power spectrum of musical notes instead of frequencies, according to the musical scale based on 13.7 Hz=A-1 note as described in previous sections. Spectral power values of all the A note (A-1, A0, A1, A2, A3, A4, A5, A6), and similarly all the A#, B, C, C#, D, D#, E, F, F#, G, G# notes are averaged and the variability of all those average values is established. The less the variability of power for each note, or in other words a good measure of power for all the musical notes, the more harmonic is the particular voice measurement. Harmonic voice measurement does not present “starvation notes” where a power in one or more notes is much lower than the rest. In order to measure the musical note variability, the following parameters are calculated by the voice analyzer: mean values of each note (A, A#, B, C, etc.) (Mean), power averages (Avg) and standard deviations (StDev) of all those averages are calculated. To quantify the harmony level of the voice measurement, the voice analyzer uses equation 3: Mean/StDev.

### Relative Power Analysis

In this analysis, a musical scale of frequencies is established by: (a) identifying first the maximal power peak in the logarithmic values of the FFT spectrum values, and (b) adding and subtracting semitone values of 12^{th} root of 2 (12√2≈1.05946) of an Equaled Tempered Scale, as explained in a previous section. As a result of this stage, a full musical scale of frequencies is built around the frequency with the maximal power (first Formant F1) of the logarithmic values of the FFT spectrum values. This calculated musical scale covers frequencies from 160 Hz (Note E2) to 1960 Hz (Note B6). Next, the voice analyzer finds the power amplitude of the frequency values nearest to the actual frequencies in the logarithmic values of the FFT spectrum values and calculate the average power value of all the frequencies closest to the F1-attuned musical scale. This average value represents the internal music harmonic power of the voice measurement.

Similarly, another musical scale herein referred to as “Dis-harmonic Musical Scale”, is built by adding and subtracting the same maximal power (first Formant F1) of the logarithmic values of the FFT spectrum values to and from a ‘non-semitone value’ which equals to=2\hat{ }(1/11.5)≈1.06213. That musical scale represents a disharmonic musical scale of frequencies because the semitone distribution does not comply with the construction of the Equaled Tempered Scale.

An “Harmonic Musical Scale” is also created. The voice analyzer recognizes the power amplitude of the frequency values nearest to the actual frequencies in the logarithmic values of the FFT spectrum values and calculate the average power value of all the frequencies closest to the F1-attuned musical scale. This average value represents the internal harmonic musical power of the voice measurement. The ratio between the average power of the Harmonic Musical Scale and the Dis-Harmonic Musical Scale values is a marker of internal vocal harmony characterizing the voice measurement.

### (2C) Golden Proportion Analysis:

The voice analyzer in this stage is configured to identify and quantify the level of the vocal harmony in the voice measurement based on the golden proportion.

The term ‘golden proportion’ refers to a constant ratio Φ=1.6180339887498948420 or to its reciprocal 1/Φ=0.6180339887498948420. The golden ratio is being used to analyze the proportions of natural objects or mathematic problems. In fact, the Φ ratio can be identified in various areas, such as geometry, mathematics, nature, architecture, arts and life science. It is considered to be a proportion that describes the concept of harmony. The golden proportion is also reflected in music. Musical scales are based on harmonics that are created by frequencies that conform to the golden proportion.

The voice analyzer employed in the disclosed invention, is configured to calculate whether the golden proportion is applied to the frequency peaks (formants) of the FFT spectrum values and to detect all frequency peak values that correspond thereto. For example, the ratio between two optional peaks, such as 286.19 Hz and 178.09 Hz is 1.607 which is close enough to Φ, therefore shows a match to the golden proportion. A deviation of up to 20% from Φ defines a close enough value, and therefore considered to be a match to the golden proportion. The voice analyzer then stores the data of all the above matches for further harmony analysis.

In some embodiments of the invention, the above golden proportion analysis is performed on logarithmic values of the FFT spectrum values.

The more such matches are found in the voice measurement, the level of the vocal harmony detected in the voice measurement is greater degree and thereby the voice analyzer enables to establish the degree of adaptation of the user's voice to the environmental.

In this sage, the voice analyzer is configured to calculate three parameters: (1) the number of incidences in which the following condition is fulfilled—frequency difference between any two peaks of frequencies is less than 20% (Diff Nr); (2) The average value of the power of the logarithmic values of the FFT spectrum values is less than 20% (Diff Pwr); (3) the result of Diff Nr*Diff Pwr. The integration of the last three parameters define the golden proportion marker for the harmony level in the voice measurement.

### Comparison and Feedback

In this stage, the voice analyzer is configured to unify all the results of the above listed analyses: entropy analysis, musical analysis, and golden proportion analysis, into one parameter.

According to some embodiment of the invention, the results of each analysis (entropy analysis; musical analysis; and golden proportion analysis) are normalized to transform them into one parameter P(analysis)ᵢ that refers to P(entropy)ᵢ or P(musical)ᵢ or P(goldenp)ᵢ, respectively, according to equation 4: P(analysis)ᵢ=(AVERAGE−MIN)/(MAX−MIN), wherein i refers to the index number of the voice measurement taken. The value of each parameter ranges from 0 to 1. In some embodiments of the invention, this P(analysis)ᵢ calculation is based on the second quartile median (Q2) instead of the average in order to avoid giving mathematical weight to unrepresentative data that can appear as a result of a particularly faulty, noisy or artefactual recordings in one of the recordings of the voice measurements. Then, said normalized values (P(entropy)ᵢ, P(musical)_{I}, P(goldenp)ᵢ) are averaged into a single parameter (Pre-Pᵢ). This is repeatedly performed in connection with all the voice measurements taken before the emotional treatment, thereby receiving several parameters, each for a single pre voice measurement: Pre-P1, Pre-P2, Pre-P3 . . . that refer to pre-voice measurement 1, 2, 3 . . . .

The same is performed with regard to all the voice measurements taken after the emotional treatment. Namely the voice analyzer normalizes and unifies all the results of the above listed analyses: entropy analysis musical analysis and golden mean analysis into one parameter (Post-Pᵢ) that its value ranges from 0 to 1, thereby receiving several parameters, each for a single post voice measurement: Post-P1, Post-P2, Post-P3 . . . that refer to post-voice measurement 1, 2, 3 . . . .

Next, the voice analyzer is configured to further unify the parameters of all voice measurements (Pre-P1, Pre-P2, Pre-P3 . . . or Post-P1, Post-P2, Post-P3 . . . ) into one parameter. At this stage, two parameters are received—Pre-P and Post-P. The first one represents the degree of harmony from the voice analysis of the user's voice before the emotional treatment and the second represents the harmony from the voice analysis of the user's voice after the emotional treatment.

Lastly, according to some embodiments of the invention, the % GAIN (% G) is calculated from the ratio between the two parameters Pre-P and Post-P according to equation 5: % G=(1−(Post-P/Pre-P))*100. The percentage of the above ratio is reflecting the shifts in harmony in the user's voice, thereby reflecting the shift in the user's mood after the emotional treatment; if % G is positive it means that there was an increase in voice harmony of the pre-treatment voice measurements as opposed to the post-treatment voice measurements, and if % G is negative it means that there was a decrease in voice harmony.

The software presents a feedback to the user, based primary on the results of % Gain of the voice analyzer. According to some embodiments of the invention, the feedback is based on both of the user's elections through the emotional treatment and the voice analysis. Examples of feedback wordings that may be used are: “NO IMPROVEMENT, MINIMAL IMPROVEMENT, REGULAR IMPROVEMENT, BIG IMPROVEMENT, AMAZING IMPROVEMENT”.

These statements are displayed according to a T-Test analysis of statistical significance to establish the significance between all the Pre-P1, Pre-P2, Pre-P3 . . . and Post-P1, Post-P2, Post-P3 . . . from their averages and related standard deviations. Positive % Gain value indicates the assessed improvement but the T-Test p parameter establishes how significant is that improvement. T-Test refers herein to a single tailed statistical test that enables to establish how significant is the difference between two sets of data. The T-Test is calculated using the Average and Standard Deviation, using equations as known in the art, and the result of the test is p (Probability). For example, p=0.5 indicates that the probability of difference is 50%, therefore there the is no significant differences between the two sets of data; p=0.05 indicates that there is a 95% of probability that there is a difference and p=0.005 that there is a 99.5% of probability that there is a difference. In view of that, if a partial improvement is occurred due to a huge increase in one particular parameter in a single stage of the analysis without a solid increase in others, the result of the T-test will clarify that the improvement was not really significant.

The software further displays to the user the above feedback wordings, optionally followed by the numerical outcome of the voice analyzer according to the T-Test results in the below table:

| | |
|---|---|
| p > 0.25 | NO IMPROVEMENT |
| 0.15 > p < 0.25 | MINIMAL IMPROVEMENT |
| 0.05 > p < 0.15 | REGULAR IMPROVEMENT |
| 0.005 > p < 0.05 | BIG IMPROVEMENT |
| p < 0.005 | AMAZING IMPROVEMENT |

At this stage, the software analyses the differences between the voice measurements before and after the emotional treatment. According to the methodology in the base of this invention, when the conscious speech mechanism is activated, the harmonic parameters of the user's voice are changed. The software enables to identify whether a user successfully went through the whole process and improved his state of mind resulting, for example, in improving its verbal communication skills, improving his ability to regain control over his intense emotions, improving his ability to manage emotional disorders, etc., based on the analysis of the user's voice measurements. The software, according to this embodiment, allows to objectively produce a customized feedback to the user about his state of mind, without requiring any direct input based on the user's election.

According to other embodiments of the invention, the % G can be further developed into a result that take into consideration its level of significance. The % Weighted Gain, (% WG), is then calculated as follows: % WG=% G*W, wherein W is the weight factor that depends on the T-Test p parameter, as follows:

| | |
|---|---|
| p > 0.25 | W = 0.1 |
| 0.2 > p < 0.25 | W = 0.3 |
| 0.15 > p < 0.2 | W = 0.5 |
| 0.1 > p < 0.15 | W = 0.7 |
| 0.05 > p < 0.1 | W = 0.8 |
| 0.01 > p < 0.05 | W = 0.9 |
| p < 0.01 | W = 1 |

For example, if the % G is +20%, namely, the voice measurement after the emotional treatment is 20% more harmonic than before, but the p value is 0.25, namely, 75% of possibility, which is of low significance, the % WG is 20%*0.1=2%. Similarly, if p<0.05 namely, 99.5% of possibility, which is of high significance, the % WG is 20%*0.8=16%. These results are much more reliable than the pure % G.

In some embodiments of the invention, the software further requests the user's input describing his current emotions, during and/or at the end of the emotional treatment in order to complete the analysis. In this embodiment, the software engages the user's input together with the objective analysis based on the user's voice measurements in order to generate a customized feedback. It is expected to obtain high correlation between the results of the user's voice measurement analysis and the user's emotions as he expresses.

As above mentioned, the software presents to the user a start screen, acknowledging the user has an actual emotional problem that he would like to solve with the help of the software. The software displays to the user a list of common emotional situations to help the user describe his current state of mind. This selection is referred to as “Linguistic Environment” (LE).

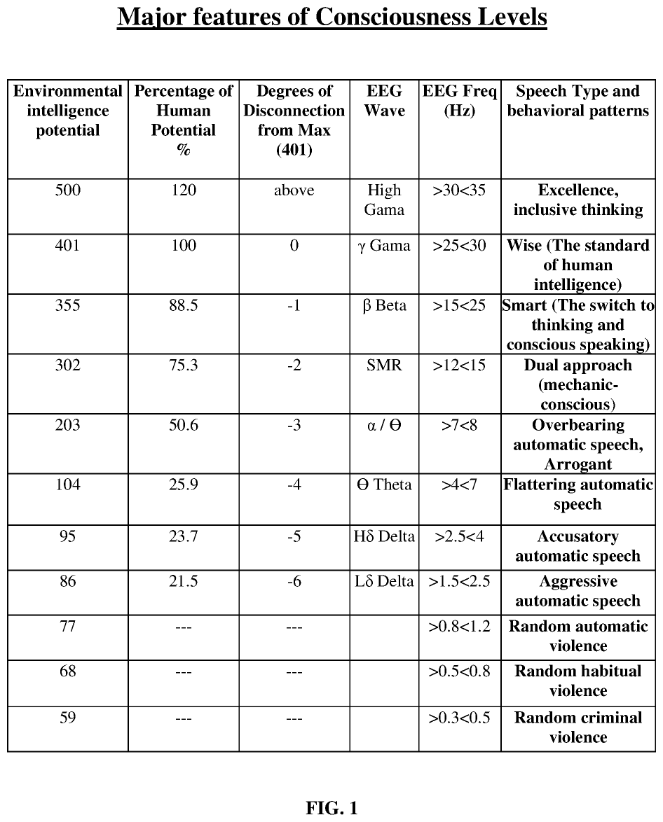

Now, referring to FIG. 1 and according to some embodiments of the invention, LE describes four main types of emotional conditions and automatic speech: (1) AGGRESSIVE AUTOMATIC SPEECH represented by an archetypal sentence “I am afraid and aggressive”; (2) ACCUSATORY AUTOMATIC SPEECH represented by an archetypal sentence “I am angry, hostile and blame others”; (3) FLATTERING AUTOMATIC SPEECH represented by an archetypal sentence “I blame myself I am a pleaser”; and (4) OVERBEARING AUTOMATIC SPEECH represented by an archetypal sentence “Nobody controls me, I decide”.

For example, a selection of a ‘FRIGHTENED’ indicates a (−6) “Level of Disconnection” (LD) and represent a situation in which only a 21% of the “Percentage of Maximal Potential” (MP). Similarly, a (−5) LD describes a situation in which 23.7% of MP is displayed. In the same way the relations between LD and MP are as follows: (−4) LD=25.9% MP; (−3) LD=50.6% MP; (−2) LD=75.3% MP; (−1) LD=88.5% MP; and (0) LD=100% MP. The user's selection defines the LD value, and the corresponding MP represents the possible gap in percentage to be gained.

The % WG can be expressed as a transit in the LD scale before and after the emotional treatment. As mentioned, each LE correlates with a suitable LD and MP. According to some embodiments of the invention, after the LE is defined the users is requested to define his level of discomfort according to a “Wellness Scale Table” (T) in a scale of 1 to 10, wherein T=1 refers to maximal discomfort and T=10 maximal wellness. T values are also correlate with said percentage value of maximal protentional of human intelligence (MP).

By choosing his LE and T the user defines quantitatively his particular emotional situation before the emotional treatment: LEpre & Tpre. Tpost is also established subjectively by the user after the emotional treatment. Each T value pre and post represent a particular MPT as shown in FIG. 1, namely: T1 to T3, MPT=21.5%; T4, MPT=23.7%; T5, MPT=25.9%; T6, MPT=23.7%; T7, MPT=50.6%; T8, % MPT=75.3%; T9, % MPT=88.5%; T10, % MPT=100%. MPT is an objective parameter of psychometric value. The total subjective psychometric assessment of the user is evaluated by the “Difference of MPT post-pre” (Diff % MPT) calculated by equation 6: Diff % MPT=% MPTpost−% MPTpre.

According to some embodiments of the invention, the software disclosed herein, further calculates an objective voice parameter called “MPT Weighted Gain” (MPTWG). MPTWG is a parameter designed before and after the emotional treatment based on Diff % MPT parameter. MPTWG is calculated using equation 7: MPTWGT=% WG*Diff % MPT/100.

According to some embodiments of the invention, the software disclosed herein, uses a comprehensive parameter that enables to evaluate the degree of improvement after the emotional treatment. That comprehensive parameter takes into consideration both the MPT parameter and the MPTWEGT as calculated according to equation 7. The “Switch My Mind % Gain” (SMM % G) is calculated using equation 8: SMM % G=Diff % MPT+MPTWGT)/2.

As mentioned above the SMM % G parameter reflects the proportional improvement of the emotional state of a user utilizing a voice analyzer that discloses sub-conscious and unconscious trend in the mind of the user, and in some embodiments of the invention, in addition to his conscious subjective assessment as reflected by the user's elections.

According to another embodiment of the invention, wherein the software is not configured to initiate an emotional treatment, but to analyze a voice measurement to evaluate a user's state of mind, the software is configured to normalize the results of each analysis described above (entropy analysis; musical analysis; and golden proportion analysis) to transform them into one parameter P(analysis)ᵢ that refers to P(entropy)ᵢ or P(musical) or P(goldenp)ᵢ, respectively in a similar manner as the one described above. In this embodiment, the software compares the user's parameters to another voice recording with similar parameters stored in the software's database. The comparison is calculated based on the third quartile (Q3) values of a general database of parameters (“Big Data”). That database is created based on voice analysis of voice measurements of variety of users in the population. The Q3 represents a mean standard value point. A Q3 is used and not the average of the parameters because the Q3 represent a value that reflects the value placed in the 75% place from all the data and that represents a fair challenge. Negative values of “Big Data % Gain” (similar to SMM % G in previous embodiment), indicate that the harmony of the user's voice is below the harmony calculated in respect to 75% of the population.

According to another embodiment of the invention, wherein the software is not configured to initiate an emotional treatment, but to analyze a voice measurement of one user alone compared with the same user in the presence or under the effect of another person, referred to herein as AFFECTING PERSON (for instance holding hands, hearing his/her voice for several seconds/minutes or looking closely at his/her eyes). In this embodiment, the software requests the user to record several voice measurements—with and without the presence of the AFFECTING PERSON. The software then analyses the differences between the voice measurements with and without the presence of the AFFECTING PERSON according to the same stages described herein above. It is expected that the harmonic parameters of the user's voice are changed.

The software of the invention is operable from any suitable electronic device, computer, computer system or related group of computer systems known in the art. In one embodiment, the software is installed upon a server or server computer system which is connected by at least one input/output port to a communication network. The communication network may be a local area network connecting a plurality of computers via any suitable networking protocol, including but not limited to Ethernet. In another embodiment, the communication network is the Internet and the system comprises server software capable of communicating with client computers via the Internet via any suitable protocol, including but not limited to HTTPS. In such case, the invention may be provided to a user as software as a service (SaaS) which will obviate a user from hardware needs such as a server and necessary server maintenance, security, etc. In one embodiment, a user may use a browser such as Internet Explorer, Mozilla Firefox, Chrome or Safari, to browse on the server via the internet. Any processing device may be utilized, including for instance, a personal computer, a laptop, a PDA or a cellular phone.

Suitable processors for implementation of the invention include, by way of example, both general and special purpose microprocessors. Generally, a processor will receive instructions and data from a read-only memory and/or a random-access memory and execute the software's instructions. The processor further controls other peripheral devices such as a touchscreen display, a screen, a keyboard, an antenna, a speaker and a microphone at the direction of the software instructions. The software is further operable to receive notice and react to user inputs, such as actuation of the touchscreen or keyboard of a desk computer.

Generally, a computer will include one or more mass storage devices for storing data files; such devices include magnetic disks, such as internal hard disks and removable disks; magneto-optical disks; and optical disks. Storage devices suitable for tangibly embodying software instructions and data include all forms of non-volatile memory, including by way of example semiconductor memory devices, such as EPROM, EPROM, and flash memory devices; magnetic disks such as internal hard disks and removable disks; magneto-optical disks; and CD-ROM disks.

The invention is embodied in any suitable programming language or combination of programming languages. Each software component can be implemented in a high-level procedural or object-oriented programming language, or in assembly or machine language if desired. The programming language may be a compiled or interpreted language.

Having thus described exemplary embodiments of the invention, it will be apparent that various alterations, modifications, and improvements will readily occur to those skilled in the art. Alternations, modifications, and improvements of the disclosed invention, though not expressly described above, are nonetheless intended and implied to be within spirit and scope of the invention. Accordingly, the foregoing discussion is intended to be illustrative only; the invention is limited and defined only by the following claims and equivalents thereto.

We claim:

1. A computerized method for analyzing and evaluating a physiological state of at least one user by operating a voice analysis system, the voice analysis system comprising a voice analyzer processor, said method comprising:
a) tuning a user's voice by playing at least one beep sound followed by silence;
b) receiving and recording at least one voice measurement from said user;
c) utilizing said voice analyzer processor for automatically analyzing said voice measurements to evaluate physiological state of said user; wherein said voice analysis is based on harmonic analysis of said voice measurements; and
d) providing said user, by visually indicating upon a display, with a feedback related to said physiological state of said user.

2. The method of claim 1, wherein adjusting said beep sound to a frequency that is associated with a natural resonance.

3. The method of claim 1, wherein each said voice measurement is recorded if the decibel level of said user's voice is at least 70 dB.

4. The method of claim 1, further comprising prior to step a), automatically detecting the ambient background noise level and setting a threshold intensity of said voice measurement to be at least 0.5 dB above said ambient background noise level.

5. The method of claim 1, wherein said recording of each said voice measurement is at least 0.25 seconds.

6. The method of claim 1, wherein analyzing said voice measurements for evaluating said physiological state of said user comprises performing harmony analysis of said voice measurements, based on at least one natural resonance or any combination thereof.

7. The method of claim 1, operable offline or through a network comprised of the internet or a local area network, wherein said user input of said voice measurement, is received from said user electronic communication device selected from: a cellular phone, a personal computer, a tablet, and a personal digital assistant.

8. The method of claim 1, wherein said user refers to a plurality of users; said method comprises receiving and recording at least one voice measurement that is composed from a plurality of sounds, that are simultaneously produced by said users as a group of people, thereby evaluating a physiological state of said users together.

9. The method of claim 1, wherein analyzing said voice measurements for evaluating said physiological state of said user under the effect of the presence of another person; wherein said steps are performed without the influence of another person, now with the influence of said person, said method further comprises:
a) tuning user's voice by playing at least one beep sound followed by silence;
b) receiving and recording at least one new voice measurement from said user;
c) utilizing said voice analyzer processor for automatically analyzing said new voice measurements to evaluate physiological state of said user; wherein said voice analysis is based on harmonic analysis of said new voice measurements;
d) comparing said evaluations of said voice measurements recorded with and without said person's presence, and that were processed by said voice analyzer processor and based on said comparison, evaluating the influence said person has on said user; and
e) providing said user, by visually indicating upon a display, with a feedback related to said influence of said person on said user.

10. The method of claim 1, wherein said user refers to two users; said method further comprises:
a) tuning second user's voice by playing at least one beep sound followed by silence;
b) receiving and recording at least one voice measurement from said second user;
c) utilizing said voice analyzer processor for automatically analyzing said voice measurements of second user to evaluate physiological state of said second user; wherein said voice analysis is based on harmonic analysis of said voice measurements;
d) comparing said evaluations of said voice measurements of said two users that were processed by said voice analyzer processor and evaluating the degree of complementarity and harmony between said two users, based on said comparison; and
e) providing said users, by visually indicating upon a display, with a feedback related to said complementarity and harmony degree.

11. The method of claim 1, wherein said method is a computerized method for further providing an computerized emotional treatment to a user by operating a voice analysis system, the voice analysis system comprising a voice analyzer processor for further evaluating a physiological state enhancement in at least two different events, said method comprising:
a) receiving a user's input indicating his pre-treatment physiological state, that is selected from a pre-defined list of optional physiological states;
b) tuning a user's voice by playing at least one beep sound followed by silence;
c) receiving and recording at least one voice measurement from said user;
d) utilizing said voice analyzer processor for automatically analyzing said voice measurements to evaluate pre-treatment physiological state of said user;
e) automatically initiating said computerized emotional treatment, that is adjusted to said pre-treatment physiological state, by retrieving media files from a database of media files related to said computerized emotional treatment;
f) further tuning said user's voice by playing at least one beep sound followed by silence;
g) receiving and recording at least one new voice measurement from said user;
h) further utilizing said voice analyzer processor for automatically analyzing said new voice measurements to evaluate post-treatment physiological state of said user; wherein said voice analysis is based on harmonic analysis of said voice measurements;
i) comparing said evaluations of said pre-treatment physiological state and said post-treatment physiological state and evaluating said physiological state enhancement based on said comparison; and
j) providing said user, by visually indicating upon a display, with a feedback related to said physiological state enhancement;
wherein the purpose of said computerized emotional treatment is to improve said physiological state of said user.

12. The method of claim 11, further comprising after step a) receiving a user's first input referring to a level of said user's feeling of discomfort, that is selected from a pre-defined list, in numerical values from 1 to 10; after step f) receiving a user's new input referring to a level of said user's feeling of discomfort, that is selected from a pre-defined list, in numerical values from 1 to 10; wherein each said value correlates with a percentage value of maximal protentional of human intelligence; and after step i) comparing said first and new inputs and evaluating said physiological state enhancement based on said comparison.

13. The method of claim 11, wherein the computerized emotional treatment is provided in response to either said input from the user; or to said evaluation of said pre-treatment physiological state as processed by said voice analyzer processor; or in response to any combination thereof.

14. The method of claim 11, wherein the comparison stage is performed multiple times, taking into account voice measurements that are received in different events including before, during or after said computerized emotional treatment stage, evaluating physiological state enhancement based on each said comparison; and providing said user, by visually indicating upon a display, with at least one feedback related to said physiological state enhancement of each said comparison.

15. A voice analyzer processor for processing and implementing a computerized method for performing a harmony analysis of a voice measurement, said voice analyzer is configured to:
a) receive and record at least one voice measurement from at least one user;
b) define a sampling rate for each voice measurement;
c) automatically calculate fast Fourier transform (FFT) spectrum values associated to each voice measurement, based on said sampling rate;
d) perform plurality of corresponding calculations, thereby said voice analyzer is further configured to:
i) automatically calculate an entropy value that is characterising said FFT spectrum values, based on probability analysis;
ii) construct harmonic frequency based on peak values of said FFT spectrum values; construct dis-harmonic frequency based on frequency peak values of said FFT spectrum values; and, automatically calculate variability of said harmonic or dis-harmonic frequency averaged values; or automatically calculate the ratio between said harmonic frequency averaged value and said dis-harmonic frequency values;
iii) identify correspondences between said frequency peak values of said FFT spectrum values or their average value, to the golden proportion, in a deviation of up to 20% in reference to the golden proportion;
e) separately normalize the results of each said corresponding calculations into one parameter ranging from 0 to 1, relating to each voice measurement; and
f) unify said parameters, each refers to each voice measurement, into one final parameter; wherein said final parameter is designed to characterize a harmony degree in said voice measurements of said user, thereby reflecting user's physiological state.

16. The voice analyzer processor of claim 15, wherein said sampling rate is calculated based on a natural resonance.

17. The voice analyzer processor of claim 15, wherein said FFT spectrum values are calculated based on a filtered voice measurement; wherein said filtered voice measurement is defined by omitting recognizable formants and elevated peaks of frequency in said voice measurement and replacing them by their mean value.

18. The voice analyzer processor of claim 15, is further configured to filter said FFT spectrum values by disregarding FFT spectrum values that exceed their mean value or by omitting all FFT spectrum values that exceed their mean value and replacing them by said mean value.

19. The voice analyzer processor of claim 18, wherein said filtration is performed on logarithmic values of said FFT spectrum values.

20. The voice analyzer processor of claim 15, is further configured to automatically calculate a logarithm of each of the FFT spectrum values.

21. The voice analyzer processor of claim 20, is further configured to automatically calculate statistical parameters based on said logarithmic values of said FFT spectrum values, including: average, quartiles, standard deviations and ratio values of any combination thereof.

22. The voice analyzer processor of claim 21, is further configured to create a general database, storing said statistical parameters of variety of users, for further analytic and statistical purposes; and comparing said statistical parameters of said user with corresponding statistical parameters stored in said general database.

23. The voice analyzer processor of claim 15, wherein said user refers to two users, thereby said voice analyzer processor is further configured to:
calculate two separate final parameters, one referring to one user and the other referring to a second user; and
calculate the ratio between said two final parameters, wherein said ratio is designed to characterize a degree of complementarity and harmony between said two users.

24. The voice analyzer processor of claim 15, for further evaluating a physiological state enhancement of a user in at least two different events, based on a harmony analysis of a voice measurement, said voice analyzer processor is further configured to:
separately normalize the results of each said corresponding calculations into one pre-parameter ranging from 0 to 1, relating to each voice measurement recorded before an emotional treatment, as initiated by said computerized method;
separately normalize the results of each said corresponding calculations into one post-parameter ranging from 0 to 1, relating to each voice measurement recorded after an emotional treatment, as initiated by said computerized method;
unify said pre-parameters, each refers to each said voice measurement recorded before said emotional treatment, into one pre-final parameter; wherein said pre-final parameter is designed to characterize a harmony degree in said voice measurements of said user recorded before said emotional treatment, thereby reflecting user's physiological state before said emotional treatment;
unify said post-parameters, each refers to each said voice measurement recorded after said emotional treatment, into one pre-final parameter; wherein said post-final parameter is designed to characterize a harmony degree in said voice measurements of said user recorded after said emotional treatment, thereby reflecting user's physiological state after said emotional treatment; and
calculate the ratio between said pre-final parameter and post-final parameter, wherein said ratio is designed to evaluate said physiological state enhancement.

25. The voice analyzer processor of claim 24, is further configured to:
establish a significance degree of said physiological state enhancement based on probability analysis.

26. A voice analysis system for processing, analyzing and evaluating a physiological state of at least one user, comprising:
a) a computer software, interacting with associated peripherals;
b) a communication device hosting said computer software, said computer software is configured to receive information from said user;
c) a database storing and analyzing said information;
wherein said computer software is configured to utilize a voice analyzer processor according to claim 15, wherein said voice analyzer processor is configured to process and analyze at least one voice measurement received and stored in said database; to evaluate a physiological state of said user, based on harmonic analysis of said voice measurements; and to provide said user with a feedback related to said evaluation of physiological state, displayable upon a user display.

27. The system of claim 26, operable offline or through a network comprised of the internet or a local area network, wherein said system utilizes a server in communication with said communication device, wherein said communication device may be utilized to communicate with said server; said database is configured to store and analyze said information received from said server; and wherein said server is configured to provide said feedback to said communication device for display upon a user display associated with said communication device.

28. The system of claim 26, wherein said communication device is selected from: a cellular phone, a personal computer, a tablet, and a personal digital assistant.
